(19) United States
(12) Patent Application Publication
(10) Pub. No.: US 2011/0237497 A1
(43) Pub. Date: Sep. 29, 2011

(54) COMPOSITIONS OF A V-ATPASE INHIBITOR IN COMBINATION WITH A GLUCOCORTICOID RECEPTOR LIGAND AND METHODS OF USE

(76) Inventors: HUAQIANG ERIC XU, Grand Rapids, MI (US); Yong Xu, Grand Rapids, MI (US); Yuanzheng He, Grand Rapids, MI (US)

(21) Appl. No.: 12/949,432

(22) Filed: Nov. 18, 2010

Related U.S. Application Data
(60) Provisional application No. 61/262,377, filed on Nov. 18, 2009.

(51) Int. Cl. A61K 31/56; A61K 31/7048; A61K 38/12; A61P 37/08; A61P 11/06; A61P 35/02; A61K 31/365; A61K 31/427; A61K 31/454; A61P 19/02; A61P 37/06; A61P 17/00; C12N 5/00

(52) U.S. Cl. 514/1.7; 514/171; 514/27; 514/21.1; 514/18.6; 514/19.6; 514/450; 514/365; 514/323; 435/375

(57) ABSTRACT

A composition of a glucocorticoid receptor (GR) ligand, or analog thereof, and a V-ATPase inhibitor, or analog thereof. A method for administering such composition to a cell either to increase glucocorticoid transrepression activity or to increase glucocorticoid transactivation activity in the cell. Also, a method for treating a subject having an inflammatory or auto-immune disease by administering such composition.

This application claims the benefit of provisional application Ser. No. 61/262,377 filed Nov. 18, 2009, entitled COMPOSITIONS OF V-ATPASE INHIBITOR OR AN ANALOG OF V-ATPASE INHIBITOR IN COMBINATION WITH A GLUCOCORTICOID RECEPTOR LIGAND AND METHODS OF USE, the entire contents of which is incorporated herein in its entirety.

### STATEMENT REGARDING FEDERALLY SPONSORED RESEARCH OR DEVELOPMENT

This invention was made in part with United States government support awarded by the following agency: National Institute of Diabetes and Digestive and Kidney Diseases (NIDDK) R01 grant number DK066202. The U.S. Government has a paid-up license in the invention and the right in limited circumstances to require the patent owner to license others on reasonable terms.

### BACKGROUND OF THE INVENTION

Vacuolar-type ATPases (V-ATPases) are ATP-driven proton pumps responsible for maintaining the pH difference across a vacuole membrane. The maintenance of pH difference is critical for vacuolar function. For instance, lysosomes are organelles that are central to degradation and the recycling processes. The interior of a lysosome is acidic (about pH 5) compared to the slightly alkaline cytosol (pH 7.2). This acidic environment is not only necessary for the hydrolytic activity of many lysosomal hydrolyases, but also is required for lysosomal normal function, such as fusion with other vacuoles (e.g. autophagosome). V-ATPase inhibitors are molecules that inhibit the normal function of V-ATPases (pumping protons across a vacuolar membrane) thus causing vacuole (e.g. lysosome) malfunction.

Glucocorticoid receptor (GR) is a member of the nuclear receptor superfamily. GR plays an important role in many physiologic processes, such as regulating glucose and lipid metabolism, bone development, maintenance of body salt balance and mediation of body immune response. GR exerts its physiologic roles through binding to its ligand (e.g. corticoid), then controlling target gene expression either by transcriptional activation (transactivation) or by transcriptional repression (transrepression). Based on its transrepression properties on major proinflammatory cytokines, such as TNF-alpha, IL-1β, IL-6, GR is a major target for treating anti-inflammatory and auto-immune diseases, such as rheumatoid arthritis, asthma, allograft rejection and allergic skin diseases. However, transactivation is an unwanted side effect of corticoid drugs, and long term use of corticoid-like drug may lead to diabetes, osteoporosis, skin atrophy and growth retardation.

### BRIEF SUMMARY OF THE INVENTION

The present invention includes a composition comprising a combination of (a) a V-ATPase inhibitor or an analog thereof and (b) a glucocorticoid receptor (GR) ligand or an analog thereof. The V-ATPase inhibitor may be an enantiomer, diasteromer, tautomer, or prodrug of the V-ATPase inhibitor, or a pharmaceutically-acceptable salt or hydrate of the V-ATPase inhibitor. In various embodiments, the V-ATPase inhibitor may be Bafilomycin A1, Concanamycin A, Lobatamide A; Archazoid A, Archazoid B, Salicylihalamide A, Salicylihalamide B, Oximidine I, Oximidine II, Apicularen A, INDOL0, SB 242784, Cruentaren, and Detruxin B. The GR ligand may be dexamethasone, cortisol, deacylcortivazol, fluticasone propionate, triamcinolone, or budesonide; and the composition may be a pharmaceutical composition. The GR ligand may be an enantiomer, diasteromer, tautomer, or prodrug of dexamethasone, cortisol, deacylcortivazol, fluticasone propionate, triamcinolone, budesonide, or other GR ligand, or a pharmaceutically-acceptable salt or hydrate of thereof.

The present invention also includes a method for increasing glucocorticoid transrepression activity in a cell by administering to a cell a combination of (a) a V-ATPase inhibitor or an analog thereof and (b) a GR ligand, or analog thereof, thereby increasing glucocorticoid transrepression activity in the cell. The V-ATPase inhibitor or GR ligand may be an enantiomer, diasteromer, tautomer, or prodrug, or a pharmaceutically-acceptable salt or hydrate thereof. In some embodiments of the invention, the V-ATPase inhibitor, or an analog thereof, and the GR ligand, or an analog thereof, may be simultaneously or concurrently administered; and in some embodiments this method may repress gene expression of proinflammatory cytokines TNF-alpha, IL-8, IL-6, or IL-1β.

Additionally, the present invention includes a method for increasing glucocorticoid transactivation activity in a cell by administering to a cell a combination of (a) a V-ATPase inhibitor or an analog thereof and (b) a GR ligand, or analog thereof, thereby increasing glucocorticoid transactivation activity in the cell; and in some embodiments, the V-ATPase inhibitor, or an analog thereof, and the GR ligand, or analog thereof, may be simultaneously or concurrently administered.

The present invention also includes a method for increasing glucocorticoid transrepression activity in a cell by administering to a cell a V-ATPase inhibitor or an analog thereof, thereby increasing glucocorticoid transrepression activity in the cell; and this method may repress gene expression of proinflammatory cytokines TNF-alpha, IL-8, IL-6, or IL-1β. The V-ATPase inhibitor may be an enantiomer, diasteromer, tautomer, or prodrug of the V-ATPase inhibitor, or a pharmaceutically-acceptable salt or hydrate of the V-ATPase inhibitor. Additionally, the present invention includes a method for increasing glucocorticoid transactivation activity in a cell by administering to a cell a V-ATPase inhibitor or an analog thereof, thereby increasing glucocorticoid transactivation activity in the cell; and the V-ATPase inhibitor may be an enantiomer, diasteromer, tautomer, or prodrug of the V-ATPase inhibitor, or a pharmaceutically-acceptable salt or hydrate of the V-ATPase inhibitor.

The present invention also includes a method for treating a subject having an inflammatory or auto-immune disease by administering to a subject in need thereof a combination of (a) a V-ATPase inhibitor or an analog thereof and (b) a GR ligand, or analog thereof, thereby treating the disease. In one embodiment, this method can be used to treat arthritis, asthma, lupus, allograft rejection, allergic skin disease, or leukemia. The V-ATPase inhibitor may be an enantiomer, diasteromer, tautomer, or prodrug of the V-ATPase inhibitor, or a pharmaceutically-acceptable salt or hydrate of the V-ATPase inhibitor. The GR ligand may be an enantiomer, diasteromer, tautomer, or prodrug of a GR ligand, or a pharmaceutically-acceptable salt or hydrate of thereof. In various embodiments, the V-ATPase inhibitor may be Bafilomycin A1, Concanamycin A, or Lobatamide A; the GR ligand may be dexamethasone, cortisol, deacylcortivazol, fluticasone propionate, triamcinolone, or budesonide; and the V-ATPase inhibitor, or an analog thereof, and the GR ligand, or analog thereof, may be administered simultaneously or concurrently.

The present invention also includes a method for treating a subject having an inflammatory or auto-immune disease by administering to a subject in need thereof a V-ATPase inhibitor or an analog thereof, thereby treating the disease. In one embodiment, this method can be used to treat arthritis, asthma, lupus, allograft rejection, allergic skin disease, or leukemia. The V-ATPase inhibitor may be an enantiomer, diasteromer, tautomer, or prodrug of the V-ATPase inhibitor, or a pharmaceutically-acceptable salt or hydrate of the V-ATPase inhibitor. In various embodiments, the V-ATPase inhibitor may be Bafilomycin A1, Concanamycin A, or Lobatamide.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown.

FIG. 1 is a graph showing Bafilomycin A1 synergizes with Dexamethasone (“Dex”) in GR-dex dose-response of induction on MMTV (pHHLuc) reporter in AD293 cells (GR transactivation). AD293 cells in 24 wells plate were transfected with 0.1 ng GR, 100 ng pHHLuc reporter together with 10 ng Renilla control plasmids in each well, then induced by EtOH (vehicle control), Dexamethasone (1 nM, 10 nM, 100 nM and 1 μM), with or without Bafilomycin A1 (100 nM). Dual-Luciferase values were measured by Promega standard manual.

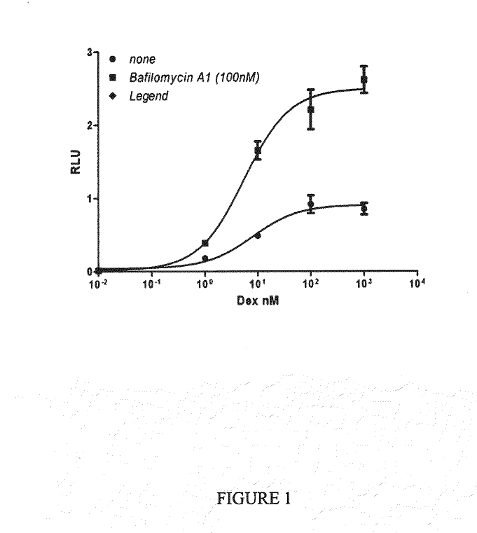

FIG. 2 is a graph showing Concanamycin A synergizes with Dex in GR-dex dose-response of induction on MMTV (pHHLuc) reporter in AD293 cells (GR transactivation). AD293 cells in 24 wells plate were transfected with 0.1 ng GR, 100 ng pHHLuc reporter together with 10 ng Renilla control plasmids in each well, then induced by EtOH (vehicle control), Dexamethasone (1 nM, 10 nM, 100 nM and 1 μM), with or without Concanamycin A (1 nM). Dual-Luciferase values were measured by Promega standard manual.

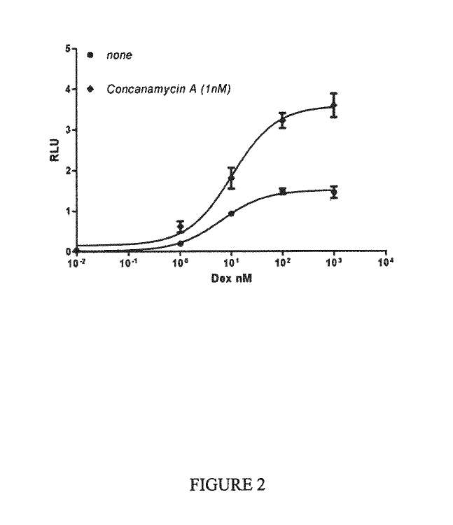

FIG. 3 is a graph showing Bafilomycin A1 enhances Dex repression on proinflammation cytokine IL-1β in macrophage THP-1 cells. THP-1 cells were first induced to differentiate into macrophage by PMA 48 hrs, then cells were induced by LPS plus EtOH (control) or Dex 100 nM, with or without Bafilomycin A1 (100 nM). 12 hrs after stimulation, extract total cell RNA and reverse transcript, and target gene (IL-1β) mRNA levels were quantified by realtime PCR.

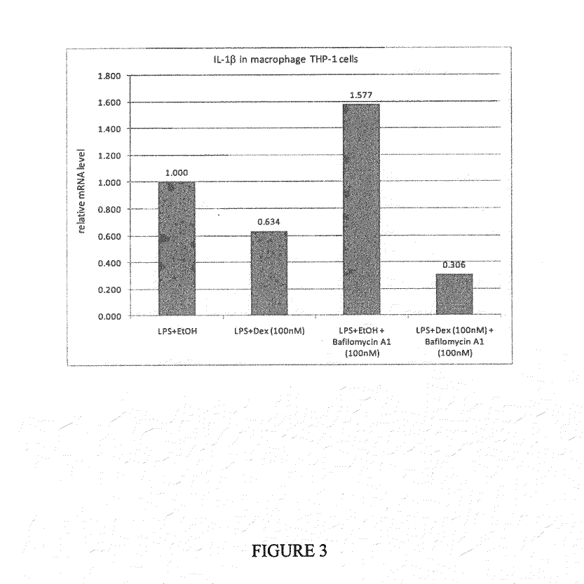

FIG. 4 is a graph showing Bafilomycin A1 enhances Dex repression on proinflammation cytokine IL-6 in macrophage THP-1 cells. THP-1 cells were first induced to differentiate into macrophage by PMA 48 hrs, then cells were induced by LPS plus EtOH (control) or Dex 100 nM, with or without Bafilomycin A1 (100 nM). 12 hrs after stimulation, extract total cell RNA and reverse transcript, and target gene (IL-6) mRNA levels were quantified by realtime PCR.

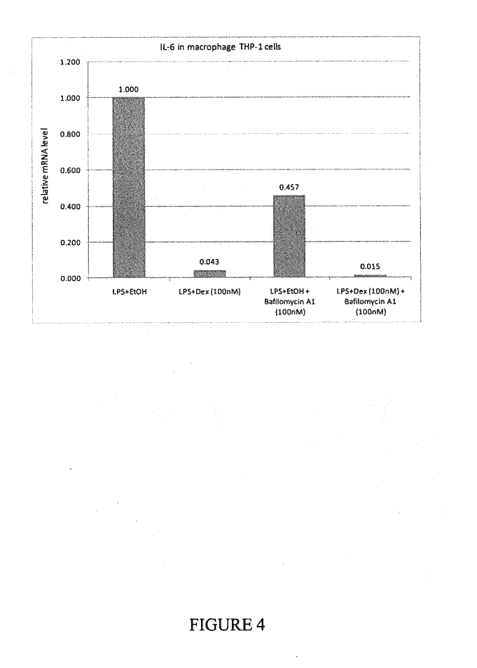

FIG. 5 is a graph showing Bafilomycin A1 synergizes with Dex in repressing proinflammation cytokine TNFα in human arthritis patient fibroblast-like synoviocytes (HFLS-RA). HFLS-RA cells were induced by TNFα, then treated with EtOH (control) or Dex 100 nM, with or without Bafilomycin A1 (100 nM). 16 hrs after stimulation, extract total cell RNA and reverse transcript, and target gene TNFα mRNA levels were quantified by realtime PCR.

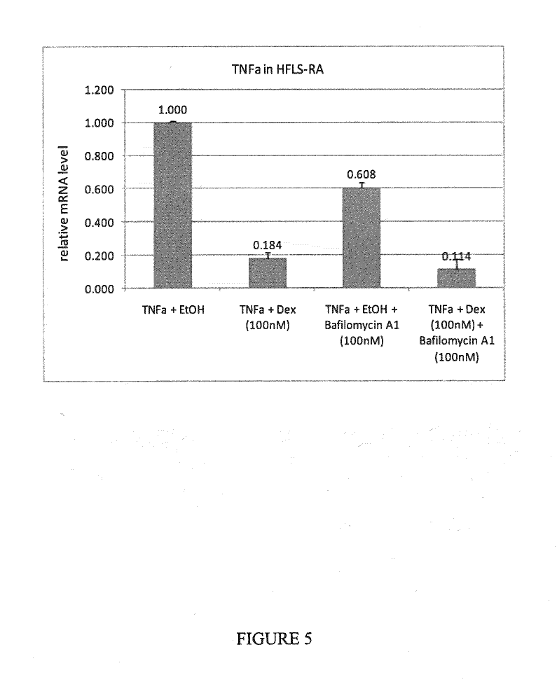

FIG. 6 is a graph showing Bafilomycin A1 synergizes with Dex in repressing matrix metallopeptidase 13 (MMP13) in human arthritis patient fibroblast-like synoviocytes (HFLS-RA). HFLS-RA cells were induced by TNFα, then treated with EtOH (control) or Dex 100 nM, with or without Bafilomycin A1 (100 nM). 16 hrs after stimulation, extract total cell RNA and reverse transcript, and target gene MMP13 mRNA levels were quantified by realtime PCR.

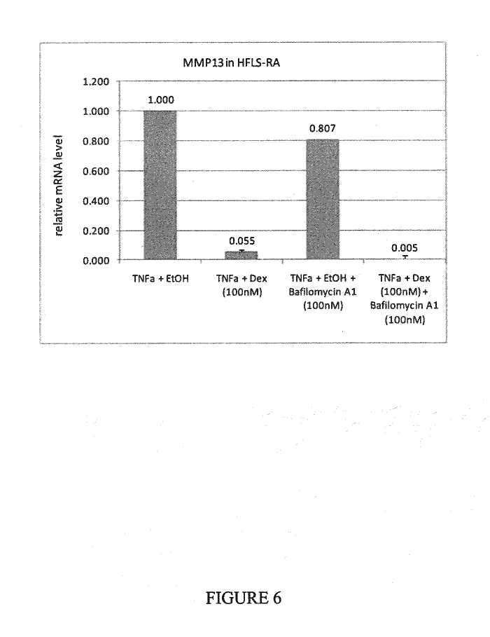

FIG. 7 is a graph showing Bafilomycin A1 synergizes with Dex in repressing COX2 in human arthritis patient fibroblast-like synoviocytes (HFLS-RA). HFLS-RA cells were induced by TNFα, then treated with EtOH (control) or Dex 100 nM, with or without Bafilomycin A1 (100 nM). 16 hrs after stimulation, extract total cell RNA and reverse transcript, and target gene COX2 mRNA levels were quantified by realtime PCR.

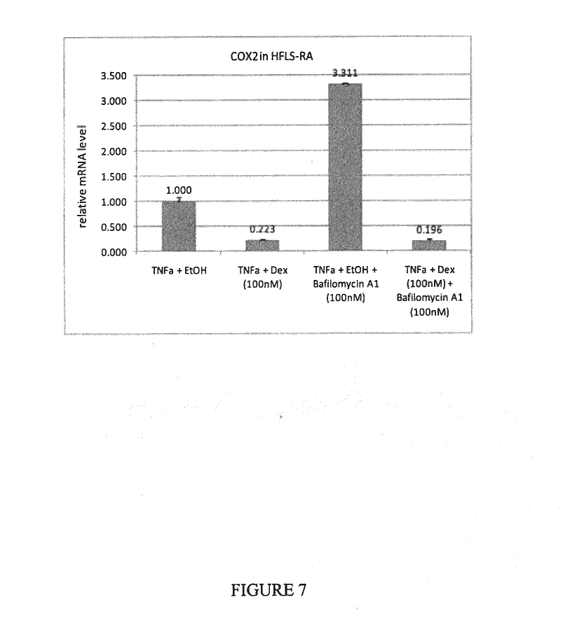

FIG. 8 is a graph showing a summary of V-ATPase inhibitors in enhancing GR-Dex activity on MMTV (pHHLuc) reporter in AD293 cells. AD293 cells in 24 wells plate were transfected with 0.1 ng GR, 100 ng pHHLuc reporter together with 10 ng Renilla control plasmids in each well, then induce by EtOH (vehicle control), Dexamethasone (10 nM), with different V-ATPase inhibitors (Bafilomycin A1 100 nM; Concanamycin A 1 nM; Lobatamide A 100 nM) or vehicle control. Dual-Luciferase values were measured by Promega standard manual. All three inhibitors showed similar enhancement of Dex-induced GR transactivation on pHHLuc.

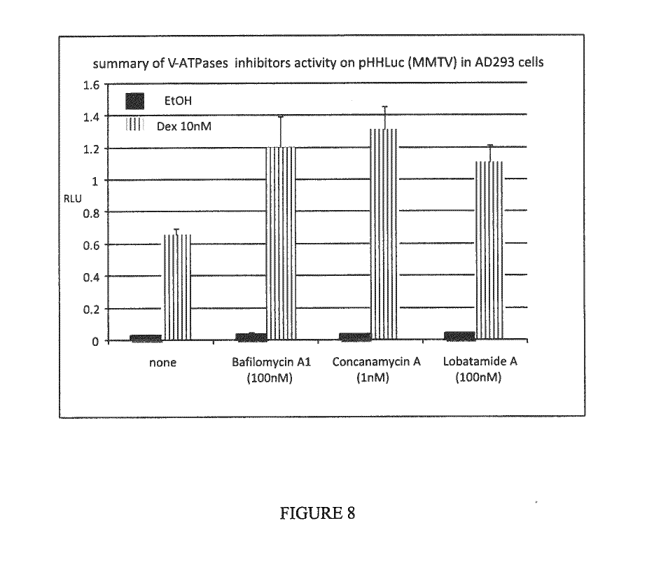

FIG. 9 is a graph showing Bafilomycin A1 enhances Dex's anti-inflammation effect in mouse collagen induced arthritis model. 7-9 weeks DBA1 mice were immunized with chicken collagen to induce arthritis. After arthritis onset, mice were IP injected with either dex (2.5 μg/mouse), Bafilomycin A (6.2 μg/mouse) or Dex and Bafilomycin A combined together. Paw swelling was measured by standard measurement of mouse clinic arthritis standard.

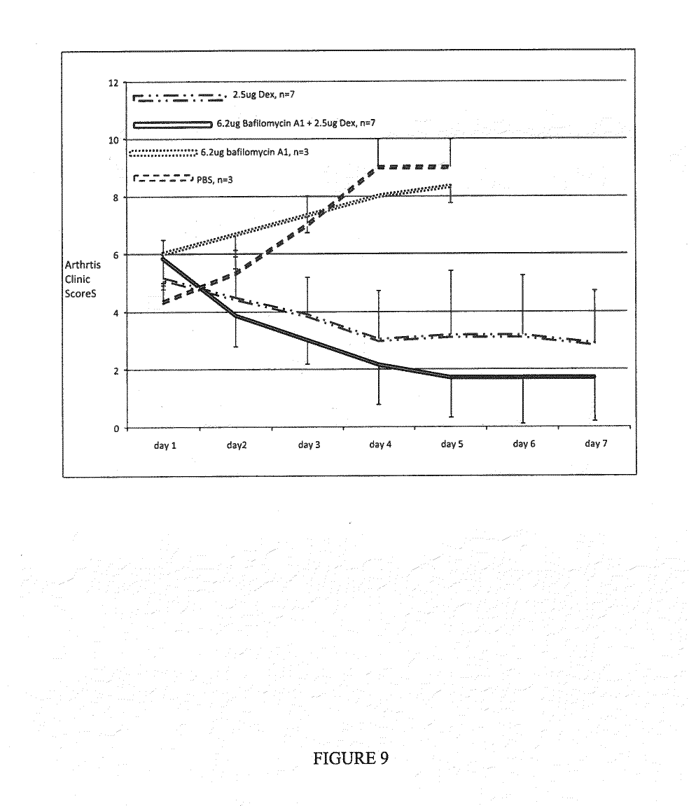

### DETAILED DESCRIPTION OF THE PREFERRED EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All references, patents, patent publications, articles, and databases, referred to in this application are incorporated herein by reference in their entirety, as if each were specifically and individually incorporated herein by reference. Such patents, patent publications, articles, and databases are incorporated for the purpose of describing and disclosing the subject components of the invention that are described in those patents, patent publications, articles, and databases, which components might be used in connection with the presently described invention. The information provided below is not admitted to be prior art to the present invention, but is provided solely to assist the understanding of the reader.

The details of one or more embodiments of the invention are set forth in the accompanying drawings and the description below. Other features, embodiments, and advantages of the invention will be apparent from the description and drawings, and from the claims. The preferred embodiments of the present invention may be understood more readily by reference to the following detailed description of the specific embodiments and the Examples included hereafter.

For clarity of disclosure, and not by way of limitation, the detailed description of the invention is divided into the subsections that follow.

Unless defined otherwise, all technical and scientific terms used herein have the meaning commonly understood by one of ordinary skill in the art to which this invention belongs. Generally, the nomenclature used herein and the laboratory procedures in cell culture, molecular genetics, organic chemistry and nucleic acid chemistry described below are those well known and commonly employed in the art. Although any methods, devices and materials similar or equivalent to those described herein can be used in the practice or testing of the invention, the preferred methods, devices and materials are now described.

In this specification and the appended claims, the singular forms “a,” “an” and “the” include plural reference unless the context clearly dictates otherwise.

As used herein the term “analogue(s)” refers to chemical compositions or compounds that are structurally similar to another but which differ slightly in composition (as in the replacement of one atom by another or in the presence or absence of a particular functional group).

The term “cell” is used herein in its usual biological sense, and does not refer to an entire multi-cellular organism. The cell can, for example, be in vitro, e.g., in cell culture, or it can be present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

As used herein, for the combination of a GR ligand/V-ATPase inhibitor combination the term “increasing glucocorticoid transactivation activity” means an increase in glucocorticoid transactivation activity in a cell as compared to the glucocorticoid transactivation activity in a cell caused by the administration of a standard GR ligand (such as Dexamethasone) alone. As used herein, for V-ATPase inhibitor without exogenous GR ligand the term “increasing glucocorticoid transactivation activity” means an increase in glucocorticoid transactivation activity in a cell as compared to the glucocorticoid transactivation activity in a cell caused by endogenous GR ligand (e.g., cortisol).

As used herein, for the GR ligand/V-ATPase inhibitor combination the term “increasing glucocorticoid transrepression activity” means an increase in glucocorticoid transrepression activity in a cell as compared to the glucocorticoid transrepression activity in a cell caused by the administration of a standard GR ligand (such as Dexamethasone) alone. As used herein, for the V-ATPase inhibitor without exogenous GR ligand the term “increasing glucocorticoid transrepression activity” means an increase in glucocorticoid transrepression activity in a cell as compared to the glucocorticoid transrepression activity in a cell caused by endogenous GR ligand (e.g., cortisol).

The term “inhibitor” as used herein with reference to an inhibitor of V-ATPase means a molecule that inhibits the normal function of a V-ATPase (e.g., pumping protons across a vacuolar membrane).

The term “ligand,” as used herein with reference to a ligand of the glucocorticoid receptor (GR), refers to a recombinant or synthetic molecule that specifically binds to GR and is suitable for use in the methods of the present invention.

The term “subject” or “patient” as used herein refers to a mammal, preferably a human, in need of treatment for a condition, disorder or disease.

As used herein, the term “therapeutically effective dose” means a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)). A “therapeutically effective dose” means an amount sufficient to prevent or reduce by at least about 25 percent, more preferably by at least 50 percent, most preferably by at least 90 percent, a clinically significant change in a feature of pathology such as for example, elevated blood pressure, fever, or white cell count, as may attend its presence and activity. As related to the present invention, the term means an amount sufficient to ameliorate, reverse, or stop the progression of one or more symptoms associated with an inflammatory or auto-immune disease.

The term “treatment” or “treating” as used herein refers to the administration of medicine or the performance of medical procedures with respect to a subject, for either prophylaxis (prevention) or to cure or reduce the extent of or likelihood of occurrence or recurrence of the infirmity or malady or condition or event in the instance where the subject or patient is afflicted. As related to the present invention, the term may also mean the administration of medicine or the performance of medical procedures as therapy, prevention or prophylaxis of an inflammatory or auto-immune disease.

Through in vitro and in vivo methods, the inventors have shown that a V-ATPase inhibitor exerts its anti-inflammatory role by potentiating the interaction of glucocorticoid and the glucocorticoid receptor. The V-ATPase inhibitor synergizes the interaction of GR and its ligands in both transactivation and transrepression, either on reporter gene assay or on endogenous GR target genes, among different types of cells. In a mouse collagen-induced arthritis model, a combination treatment of a V-ATPase inhibitor (Bafilomycin A1) and a GR ligand (dexamethasone), showed that this combination not only has a better effect than a V-ATPase inhibitor or a GR ligand alone, but also provides a way to lower steroid drug dose in such treatment, thereby avoiding the unwanted side effect of a high dose of steroid hormones.

The present invention is a composition that includes a combination of (a) a V-ATPase inhibitor or an analog thereof (e.g., the compounds shown in Examples 1-13 hereinbelow) and (b) a GR ligand; or analog thereof, e.g., Dexamethasone (Dex), cortisol, deacylcortivazol, fluticasone propionate, triamcinolone, and budesonide. This composition can be used in the treatment of auto-immune diseases (e.g., rheumatoid arthritis, lupus, asthma, skin allergy) and other diseases that are currently treated with glucocorticoid drugs (e.g. leukemia).

The inventors have identified a class of compounds that have the ability to synergize the interaction of GR and it ligands. This class includes V-ATPase inhibitors, their enantiomers, diasteromers, tautomers, prodrugs thereof, or pharmaceutically-acceptable salts, or hydrates, thereof. Several members of this class of compounds are shown in Examples 1-13 hereinbelow. This class of compounds can be used in combination with exogenous GR ligands (or analogs thereof), or without exogenous GR ligands, to synergize the interaction of GR with it ligands.

The compositions described herein can be useful for treating inflammatory or auto-immune diseases, for example, arthritis, asthma, lupus, allograft rejection, allergic skin disease, or leukemia.

The present invention encompasses all isotopes of atoms occurring in the present compounds and compositions. Isotopes include those atoms having the same atomic number but different mass numbers. By way of general example and without limitation, isotopes of hydrogen include tritium and deuterium. Isotopes of carbon include C-13 and C-14.

The compounds and compositions provided herein may contain one or more asymmetric carbon atoms and can thus exist as racemates, mixtures of enantiomers, single enantiomers, diastereomeric mixtures and individual diastereomers. All such isomeric forms of these compounds are expressly encompassed herein. Each stereogenic carbon may be independently of the R or S configuration. Although the specific compounds exemplified in this application may be depicted in a particular stereochemical configuration, compounds having the opposite stereochemistry at any given chiral center or mixtures thereof are encompassed herein. Although amino acids and amino acid side chains may be depicted in a particular configuration, both natural and unnatural forms are encompassed herein.

Compounds and compositions of the present invention also include tautomers. As used herein, the term “tautomer” means a compound or composition which is capable of existing in a state of equilibrium between two isomeric forms. Such compounds may differ in the bond connecting two atoms or groups and the position of these atoms or groups in the compound or composition.

Certain compounds or compositions of the present invention can exist in unsolvated forms as well as solvated forms, including hydrated forms. In general, the solvated forms are equivalent to unsolvated forms and are encompassed within the scope of the present invention. Certain compounds and compositions of the present invention may exist in multiple crystalline or amorphous forms. In general, all physical forms are equivalent for the uses contemplated by the present invention and are intended to be within the scope of the present invention.

The present invention also encompasses prodrugs of the compounds and compositions described herein. In general, such prodrugs can be functional derivatives of these compounds and compositions, which can readily be converted in vivo into defined compounds or compositions. Conventional procedures for selecting and preparing suitable prodrugs are known to one of ordinary skill in the art.

Also provided are pharmaceutically acceptable salts, pharmaceutically acceptable solvates, enantiomers, diastereomers, N-oxides, prodrugs, and metabolites of the compounds and compositions described herein in combination with one or more pharmaceutically acceptable carriers and optionally included excipients(s).

The term “pharmaceutically acceptable salts” refer to derivatives of the disclosed compounds and compositions which are modified by making its acid or base salts. Examples of pharmaceutically acceptable salts include, but are not limited to, mineral or organic acid salts of basic residues, for example, amines; alkali or organic salts of acidic residues, for example, carboxylic acids; and the like.

The compounds and compositions disclosed herein may be prepared by techniques well known in the art and familiar to the skilled synthetic organic chemist.

A composition of a V-ATPase inhibitor and a GR ligand is more effective than either V-ATPase inhibitor or GR ligand alone. Therefore, this composition could be used in treating some severe symptoms under conditions when either corticoid drug (e.g., dexamethasone, cortisol, deacylcortivazol, fluticasone propionate, triamcinolone, and budesonide) or V-ATPase inhibitor alone is not sufficient. Such a combination therapy may also decrease the dose of steroid drug, thus, decreasing the unwanted side effect of steroid hormone drugs. Further, using a V-ATPase inhibitor without also using an exogenous GR ligand will decrease the unwanted side effect of steroid hormone drugs yet may still be sufficient for treating auto-immune or inflammatory diseases.

As to the present composition which is a combination of a V-ATPase inhibitor, or analog thereof, and a GR ligand, or analog thereof, the administration of V-ATPase inhibitor and GR ligand may be concurrent, simultaneous, or sequential (that is, the V-ATPase inhibitor or analog thereof may be administered prior to or after the GR ligand, or analog thereof).

The present invention includes a method for increasing glucocorticoid receptor-mediated gene transcriptional control (transactivation or transrepression) comprising administering to a cell a GR ligand and a V-ATPase inhibitor. The present invention also includes a method for increasing glucocorticoid receptor-mediated gene transcriptional control (transactivation or transrepression) comprising administering to a cell a V-ATPase inhibitor without co-administering an exogenous GR ligand. For each of these methods, in one embodiment the V-ATPase inhibitor is Bafilomycin A1, Concanamycin A, Lobatamide A, or one of the other molecules shown in Examples 1-13 hereinbelow.

The present invention also includes a method for treating a subject having an inflammatory or auto-immune disease. The disease treated may be arthritis, asthma, lupus, allograft rejection, allergic skin disease, leukemia, or other inflammatory and auto-immune diseases. Such treatment includes administering to a subject having an inflammatory or auto-immune disease a GR ligand (its enantiomer, diastereomer, tautomer, a prodrug thereof, or a pharmaceutically-acceptable salt, or hydrate, thereof) in combination with a V-ATPase inhibitor, its enantiomer, diastereomer, tautomer, a prodrug thereof, or a pharmaceutically-acceptable salt, or hydrate, thereof. The V-ATPase inhibitor, its enantiomer, diastereomer, tautomer, a prodrug thereof, or a pharmaceutically-acceptable salt, or hydrate, thereof, also could be administered to the subject without co-administering GR ligand, its enantiomer, diastereomer, tautomer, a prodrug thereof, or a pharmaceutically-acceptable salt, or hydrate, thereof.

The GR ligand/V-ATPase inhibitor combination (or the VTPase inhibitor without GR ligand) used in the present method of treatment is administered parenterally, orally, topically (transdermally) or rectally. Parental administration includes intravenous (IV), intramuscular, intradermal, intraperitoneal (IP) or subcutaneously (SQ). Parenteral administration requires a sterile isotonic aqueous solution buffered to an appropriate pH for the selected compounds or a suspension or emulsion for sustained release administration.

The compositions of the present invention can be administered orally by solid dosage forms such as tablets, capsules, dispersible granules or lozenges and by liquid dosage forms such as solutions, syrups, suspensions and emulsions. These would include dosages form for immediate release as well as sustained dosage forms for 12 or 24 hour administration.

For preparing the compositions of this invention, inert, pharmaceutically acceptable carriers may be used. Those carriers can be either solid or liquid. Solid form preparations include powders, tablets, dispersible granules, capsules, cachets, suppositories and ointments. A solid carrier can be one or more substances which may also act as diluents, flavoring agents, solubilizers, lubricants, suspending agents, binders, or tablets disintegrating agents; it can also be as finely divided solid which is in admixture with the finely divided active compound. For the preparation of tablets, the active compound (or compounds) is mixed with carrier having the necessary binding properties in suitable proportions and compacted in the shape and size desired. The powders and tablets can, in some embodiments, contain from about 5 to about 70 percent of the active ingredient. Suitable solid carriers are lactose, pectin, dextrin, starch, gelatin, tragacanth, low melting wax, cocoa butter and the like. The term “preparation” is intended to include the formulation of the active compound with encapsulating material as carrier providing a capsule in which the active component (with or without other carriers) is surrounded by carrier, which is thus in association with it. Similarly, capsules can be used, as solid dosage forms suitable for oral administration.

Liquid form preparations include solutions suspensions and emulsions. As an example may be mentioned water or water-propylene glycol solutions for parenteral injection. Such solutions are prepared so as to be acceptable to biological systems with respect to isotonicity, pH, and other parameters. Liquid preparations can also be formulated in solution in aqueous polyethylene glycol solution. Aqueous solutions suitable for oral use can be prepared by dissolving the active component in water and adding suitable colorants, flavors, stabilizing, and thickening agents as desired. Aqueous suspension suitable for oral use can be made by dispersing the finely divided active component in water with viscous material, for example, natural or synthetic gums, resins, methyl cellulose, sodium carboxymethyl cellulose and other suspending agents.

Ointment preparations can contain heavy metal salts of a compound of Formula I with a physiologically acceptable carrier. The carrier is desirably a conventional water-dispersible hydrophilic or oil-in-water carrier, particularly a conventional semi-soft or cream-like water-dispersible or water soluble, oil-in-water emulsion infected surface with a minimum of discomfort. Suitable compositions may be prepared by merely incorporating or homogeneously admixing finely divided compounds with the hydrophilic carrier or base or ointment.

The pharmaceutical preparation can be in unit dosage form. In such forms, the preparation is subdivided into unit doses containing appropriate quantities of the active component (or components). The unit dosage form can be a packaged preparation, the package containing discrete capsules, powders in vials or ampoules and ointments capsule, cachet, tablet, gel, or cream itself or it can be the appropriate number of any of these packaged forms.

The compositions of the present invention can be administered by way of a transdermal patch which is of particular benefit for those unable to swallow where parenteral administration is not desirable. Further, the compositions of the present invention can be formulated into suppositories for rectal administration which is of particular benefit for those unable to swallow where parenteral administration is not desirable. It is known to those skilled in the art how to prepare the sterile parenteral formulations for parenteral administration, solid and liquid dosage forms for oral administrating, transdermal patches and suppositories of the compounds of the present invention. The compositions of the present invention also can be administered in dosage forms suitable for topical administration including, but not limited to, creams, ointments, lotions, solutions, suspensions, emulsions and bandages impregnated with the selected compounds of the present invention. It is known to those skilled in the art how to prepare the topical pharmaceutical dosage forms to administer the compounds of the present invention.

The selected compounds of the present invention are administered in an effective amount either to increase glucocorticoid transrepression activity or to increase glucocorticoid transactivation activity, as desired; and the selected compounds also are given in an effective amount to treat inflammatory or auto-immune disease. Such effective amount is from about 1 to about 100 mg/kg/day of each of GR ligand and V-ATPase inhibitor, preferably from about 5 to about 50 mg/kg/day of each of GR ligand and V-ATPase inhibitor. The dosage of GR ligand may or may not be the same as the dosage of V-ATPase inhibitor, and GR ligand may be eliminated entirely. Parenterally, these compounds can be given continuously by way of an IV one to four times daily by injection. When infused IV is used, it should be given at about 60 to 120 ml/hr depending on the concentration of the mixture being administered. Orally, the dose can be given once a day or divided into two or four doses a day. Topically, the topical formulation should have an effective amount of from about 0.05% to 5% of the selected compounds.

The order of administration of GR ligand and V-ATPase inhibitor, the exact dosages, and the frequencies of administration depend on the particular compounds used, the particular condition being treated, the severity of the condition being treated, the age, weight, general physical condition of the particular subject, other medication used by the subject, and/or the subject's response to the particular condition being treated as is known to those skilled in the art. Further, physicians can monitor the progress of treatment by monitoring blood markers as well as the blood level of the selected compounds as is known to those skilled in the art.

The subject being treated is a warm blooded mammal, including, humans, farm animals such as horses, sheep, cattle, lamas, pigs and the like, as well as pets such as cats and dogs. In one embodiment, the warm blooded mammal is a human.

Having now generally described the invention, the same will be more readily understood through reference to the following examples, which are provided by way of illustration, and are not intended to be limiting of the present invention, unless specified.

### EXAMPLES

### Example 1

### Bafilomycin A1, (3E,5E,11E,13E)-16-(4-(2,4-dihydroxy-6-isopropyl-5-methyltetrahydro-2H-pyran-2-yl)-3-hydroxypentan-2-yl)-8-hydroxy-15-methoxy-3,5,7,9,11-pentamethyloxacyclohexadeca-3,5,11,13-tetraen-2-one

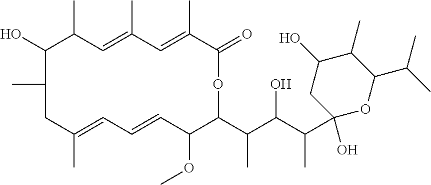

### Example 2

### Concanamycin A, 6-(2-(4-((4E,6E,14E,16Z)-11-ethyl-10,12-dihydroxy-3,17-dimethoxy-7,9,13,15-tetramethyl-18-oxooxacyclooctadeca-4,6,14,16-tetraen-2-yl)-3-hydroxypentan-2-yl)-2-hydroxy-5-methyl-6-((E)-prop-1-enyl)tetrahydro-2H-pyran-4-yloxy)-4-hydroxy-2-methyltetrahydro-2H-pyran-3-yl carbamate.

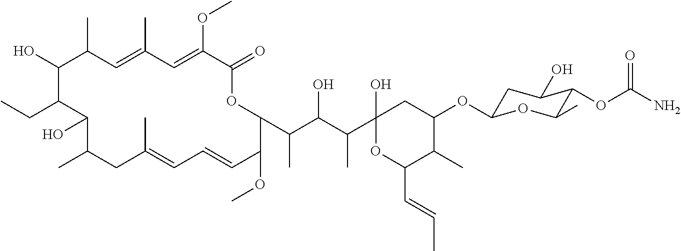

### Example 3

### Lobatamide A, (2Z,4Z)-N-((E)-3-(8E,11Z)-10,17-dihydroxy-7,11-dimethyl-1,5-dioxo-3,4,5,7,10,13-hexahydro-1H-benzo[g][1,5]dioxacyclopentadecin-3-yl)prop-1-enyl)-4-(methoxyimino)but-2-enamide

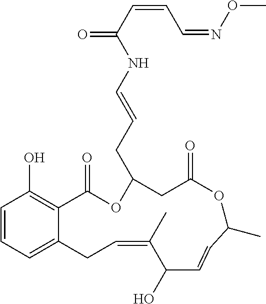

### Example 4

### Archazoid A, 1-(4-((4E,6E,11E,13Z,15Z,19E,22E)-10,18-dihydroxy-8-methoxy-3,7,9,13,15,17,20,23-octamethyl-24-oxooxacyclotetracosa-4,6,11,13,15,19,22-heptaen-2-yl)thiazol-2-yl)-3-methylbutyl methylcarbamate

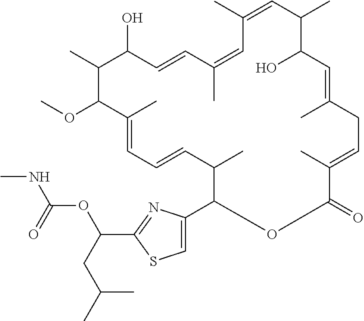

### Example 5

### Archazoid B, 1-(4-((4E,6E,11E,13Z,15Z,19E,22E)-10,18-dihydroxy-8-methoxy-3,7,9,13,15,17,20-heptamethyl-24-oxooxacyclotetracosa-4,6,11,13,15,19,22-heptaen-2-yl)thiazol-2-yl)-3-methylbutyl methylcarbamate

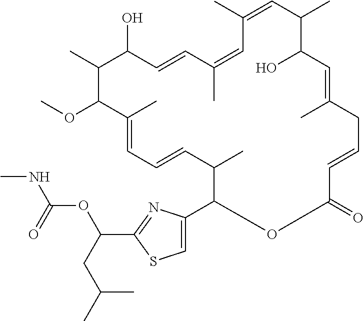

### Example 6

### Salicylihalamide A, (2Z,4Z)-N-((E)-3-((E)-5,14-dihydroxy-6-methyl-1-oxo-3,4,5,6,7,10-hexahydro-1H-benzo[c][1]oxacyclododecin-3-yl)prop-1-enyl)hepta-2,4-dienamide

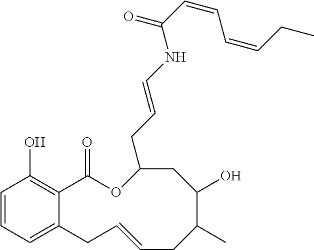

### Example 7

### Salicylihalamide B, (2Z,4Z)-N-((Z)-3-((E)-5,14-dihydroxy-6-methyl-1-oxo-3,4,5,6,7,10-hexahydro-1H-benzo[c][1]oxacyclododecin-3-yl)prop-1-enyl)hepta-2,4-dienamide

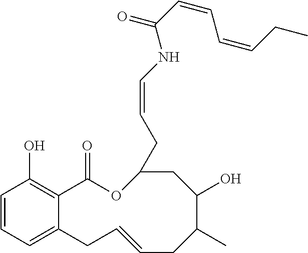

### Example 8

### Oximidine I, (2Z,4E)-N-((E)-3-((10E,12Z)-2,6-dihydroxy-5-oxo-2,3,5,13a-tetrahydro-1 aH-benzo[c]oxireno[2,3-i][1]oxacyclododecin-3-yl)allyl)-4-(methoxyimino)but-2-enamide

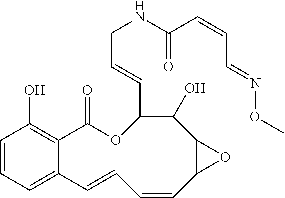

### Example 9

### Oximidine II, (2Z,4E)-N-((E)-3-((5Z,7Z,9E)-4,14-dihydroxy-1-oxo-3,4-dihydro-1H-benzo[c][1]oxacyclododecin-3-yl)allyl)-4-(methoxyimino)but-2-enamide

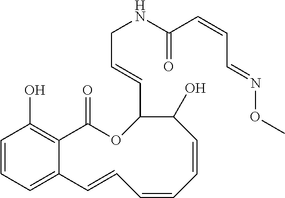

### Example 10

### Apicularen A

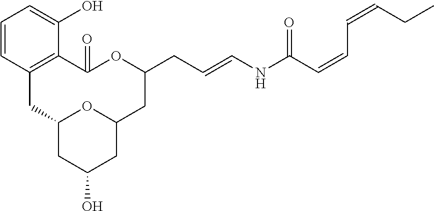

### Example 11

### INDOL0, SB 242784, (2Z,4E)-5-(5,6-dimethyl-1H-indol-2-yl)-2-methoxy-N-(1,2,2,6,6-pentamethylpiperidin-4-yl)penta-2,4-dienamide

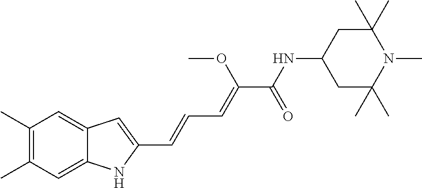

### Example 12

### Cruentaren, N-((E)-7-(E)-9,14-dihydroxy-12-methoxy-8-methyl-1-oxo-3,4,7,8,9,10-hexahydro-1H-benzo[c][1]oxacyclododecin-3-yl)-6-hydroxy-5-methyloct-2-enyl)-3-hydroxy-2-methylhexanamide

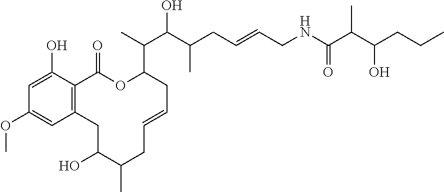

### Example 13

### Detruxin B, (3S,6S,9S,16R,21aS)-3-sec-butyl-16-isobutyl-6-isopropyl-5,8,9-trimethyldodecahydropyrrolo[1,2-d][1,4,7,10,13,16]oxapentaazacyclononadecine-1,4,7,10,14,17(1H,16H)-hexaone

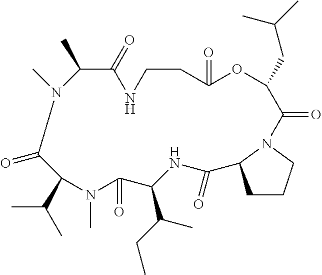

While the foregoing specification has been described with regard to certain preferred embodiments, and many details have been set forth for the purpose of illustration, it will be apparent to those skilled in the art that the invention may be subject to various modifications and additional embodiments, and that some of the details described herein can be varied considerably without departing from the spirit and scope of the invention. Such modifications, equivalent variations and additional embodiments are also intended to fall within the scope of the appended claims.

What is claimed is:

1. A composition comprising:
a) a glucocorticoid receptor (GR) ligand, its enantiomers, diasteromers, tautomers, prodrugs thereof, or pharmaceutically-acceptable salts or hydrates thereof, and
b) a V-ATPase inhibitor,
its enantiomers, diasteromers, tautomers, prodrugs thereof, or pharmaceutically-acceptable salts or hydrates thereof.

2. The composition of claim 1 wherein the V-ATPase inhibitor is selected from the group consisting of Bafilomycin A1, Concanamycin A, Lobatamide A, Archazoid A, Archazoid B, Salicylihalamide A, Salicylihalamide B, Oximidine I, Oximidine II, Apicularen A, INDOL0, SB 242784, Cruentaren, and Detruxin B.

3. The composition of claim 1 wherein the GR ligand is selected from the group consisting of dexamethasone, cortisol, deacylcortivazol, fluticasone propionate, triamcinolone, and budesonide.

4. The composition of claim 1 wherein the composition is a pharmaceutical composition.

5. A method for increasing glucocorticoid transrepression activity in a cell comprising administering to a cell the composition of claim 1, thereby increasing glucocorticoid transrepression activity in the cell.

6. The method of claim 5 wherein the administration of the composition represses gene expression of a proinflammatory cytokine selected from the group consisting of TNF-alpha, IL-8, IL-6, and IL-1β.

7. The method of claim 5 wherein the GR ligand and the V-ATPase inhibitor are simultaneously or concurrently administered.

8. A method for increasing glucocorticoid transactivation activity in a cell comprising administering to a cell the composition of claim 1, thereby increasing glucocorticoid transactivation activity in the cell.

9. The method of claim 8 wherein the GR ligand and the V-ATPase inhibitor are simultaneously or concurrently administered.

10. A method for treating a subject having an inflammatory or auto-immune disease comprising administering to a subject having an inflammatory or auto-immune disease a therapeutically effective dose of the composition of claim 1, thereby treating the disease.

11. The method of claim 10 wherein the disease is arthritis, asthma, lupus, allograft rejection, allergic skin disease, or leukemia.

12. The method of claim 10 wherein the GR ligand and the V-ATPase inhibitor are simultaneously or concurrently administered.

13. The method of claim 10 wherein the V-ATPase inhibitor is selected from the group consisting of Bafilomycin A1, Concanamycin A, Lobatamide A, Archazoid A, Archazoid B, Salicylihalamide A, Salicylihalamide B, Oximidine I, Oximidine II, Apicularen A, INDOL0, SB 242784, Cruentaren, and Detruxin B.

14. The method of claim 10 wherein the GR ligand is selected from the group consisting of dexamethasone, cortisol, deacylcortivazol, fluticasone propionate, triamcinolone, and budesonide.

15. A method for increasing glucocorticoid transrepression activity in a cell comprising administering to a cell a V-ATPase inhibitor, its enantiomers, diasteromers, tautomers, prodrugs thereof, or pharmaceutically-acceptable salts or hydrates thereof, thereby increasing glucocorticoid transrepression activity in the cell.

16. The method of claim 15 wherein the administering of the V-ATPase inhibitor, its enantiomers, diasteromers, tautomers, prodrugs thereof, or pharmaceutically-acceptable salts or hydrates thereof, represses gene expression of a proinflammatory cytokine selected from the group consisting of TNF-alpha, IL-8, IL-6, and IL-1β.

17. A method for increasing glucocorticoid transactivation activity in a cell comprising administering to a cell a V-ATPase inhibitor, its enantiomers, diasteromers, tautomers, prodrugs thereof, or pharmaceutically-acceptable salt or hydrates thereof, thereby increasing glucocorticoid transactivation activity in the cell.

18. A method for treating a subject having an inflammatory or auto-immune disease comprising administering to a subject having an inflammatory or auto-immune disease a therapeutically effective dose of a V-ATPase inhibitor, its enantiomers, diasteromers, tautomers, prodrugs thereof, or pharmaceutically-acceptable salts or hydrates thereof, thereby treating the disease.

19. The method of claim 18 wherein the disease is arthritis, asthma, lupus, allograft rejection, allergic skin disease, or leukemia.

20. The method of claim 18 wherein the V-ATPase inhibitor is selected from the group consisting of Bafilomycin A1, Concanamycin A, Lobatamide A, Archazoid A, Archazoid B, Salicylihalamide A, Salicylihalamide B, Oximidine I, Oximidine II, Apicularen A, INDOL0, SB 242784, Cruentaren, and Detruxin B.
